(19) United States
(12) Patent Application Publication
(10) Pub. No.: US 2024/0279151 A1
(43) Pub. Date: Aug. 22, 2024

(54) METHODS FOR SOLVATION AND REMOVAL OF IODINE (I2)-CONTAINING SPECIES

(71) Applicant: Honeywell International Inc., Charlotte, NC (US)

(72) Inventors: Haluk Kopkalli, Staten Island, NY (US); Daniel C. Merkel, Orchard Park, NY (US); Haiyou Wang, Amherst, NY (US); Terris Yang, East Amherst, NY (US); Richard Wilcox, West Caldwell, NJ (US); Tao Wang, Shanghai (CN); Jennifer W. McClaine, Branchburg, NJ (US); Gavin Towler, Charlotte, NC (US); Haridasan K. Nair, Wiliamsville, NY (US)

(21) Appl. No.: 18/570,428

(22) PCT Filed: Jul. 11, 2022
(86) PCT No.: PCT/US2022/073600

§ 371 Date: Dec. 14, 2023

Related U.S. Application Data
(60) Provisional application No. 63/222,811, filed on Jul. 16, 2021.

(51) Int. Cl. C07C 51/64; B01D 3/40; B01D 11/04

(52) U.S. Cl. CPC C07C 51/64; B01D 3/40; B01D 11/0492

(57) ABSTRACT

The present disclosure provides a method of removing iodine (I₂) and iodine-containing species from processes for producing trifluoroiodomethane (CF₃I). The present disclosure further provides another method of removing iodine and iodine-containing species from trifluoroacetyl iodide (TFAI).

### CROSS-REFERENCE TO RELATED APPLICATION

This application claims priority to Provisional Application No. 63/222,811, filed Jul. 16, 2021, which is herein incorporated by reference in its entirety.

### FIELD

The present disclosure relates to processes for producing trifluoroiodomethane (CF₃I). Specifically, the present disclosure relates to methods for removing iodine (I₂)-containing species from a trifluoroacetyl iodide (TFAI) feedstock used to produce trifluoroiodomethane and from a reactor effluent stream to improve the process for producing trifluoroiodomethane from trifluoroacetyl iodide (TFAI).

### BACKGROUND

Trifluoroiodomethane (CF₃I), also known as perfluoromethyliodide, trifluoromethyl iodide, or iodotrifluoromethane, is a useful compound in commercial applications, as a refrigerant or a fire suppression agent, for example. Trifluoroiodomethane (CF₃I) is an environmentally acceptable compound with a low global warming potential and low ozone depletion potential. Trifluoroiodomethane (CF₃I) can replace more environmentally damaging materials.

Methods of preparing trifluoroiodomethane are known. For example, U.S. Pat. No. 7,132,578 (Mukhopadhyay et al.) discloses a catalytic, one-step process for producing trifluoroiodomethane from trifluoroacetyl chloride. However, the source of iodine, is iodine fluoride (IF). Iodine fluoride is relatively unstable, decomposing above 0° C. to I₂ and IFs. Iodine fluoride may also not be available in commercially useful quantity.

In another example, U.S. Pat. No. 7,196,236 (Mukhopadhyay et al.) discloses a catalytic process for producing trifluoroiodomethane using reactants comprising a source of iodine, such as hydrogen iodide, at least a stoichiometric amount of oxygen, and a reactant CF₃R, where R is selected from the group consisting of —COOH, —COX, —CHO, —COOR₂, AND —SO₂X, where R₂ is alkyl group and X is a chlorine, bromine, or iodine. Hydrogen iodide, which may be produced by the reaction, is oxidized by at least a stoichiometric amount of oxygen, producing water and iodine for economic recycling. Several other processes are referenced in the literature for making trifluoroiodomethane (CF₃I) from trifluoroacetyl chloride with hydrogen iodide in a vapor phase reaction (for example U.S. Pat. No. 10,752,565).

In yet another example, U.S. patent application Ser. No. 16/549,412 (issued as U.S. Pat. No. 10,954,177) discloses a two-step process for producing trifluoroiodomethane from trifluoroacetyl chloride. The process consists of a first step of making trifluoroacetyl iodide via the reaction of CF₃COCl+HI□CF₃COI+HCl and a second step of making trifluoroiodomethane via the reaction of CF₃COI□CF₃I+CO. This process provides higher selectivity to trifluoroiodomethane (CF₃I) than others.

While developing the process, it was found that even purified trifluoroacetyl iodide (TFAI) feed material contained other iodine (I₂)-containing species, such as I₂, and HI₃. During the conversion step of trifluoroacetyl iodide (TFAI) to trifluoroiodomethane (CF₃I), the presence of iodine (I₂)-containing species, such as I₂ and HI₃, together with additional I₂ formed during this reaction as well as the reaction to produce trifluoroacetyl iodide (TFAI) from trifluoroacetyl chloride (TFAC) and hydrogen iodide (HI), caused increased corrosion of equipment and/or operational difficulties including flow, pressure control and plugging issues. These results are disadvantageous from the standpoints of reduced productivity of the desired product and an increased operational cost. Hence, there is a need for a means to remove iodine (I₂)-containing species including I₂ and HI₃ before and/or after the reaction to form trifluoroiodomethane (CF₃I).

### SUMMARY

The present disclosure provides a method for solvation and removal of an iodine (I₂)-containing species comprising the following steps: providing a feedstock comprising trifluoroacetyl iodide (TFAI) and the iodine (I₂)-containing species; adding a solvent, such as toluene, to the feedstock stream to provide a mixture comprising the solvent, trifluoroacetyl iodide (TFAI) and the iodine (I₂)-containing species; and passing the mixture to one or more columns to obtain a purified stream comprising trifluoroacetyl iodide (TFAI).

The present disclosure further provides a method of removing iodine (I₂) from a stream comprising trifluoroacetyl iodide (TFAI), and at least an iodine (I₂)-containing species selected from iodine (I₂) and HI₃. The method comprises: providing a feed stream, a solvent such as toluene, and at least an iodine (I₂)-containing species selected from iodine (I₂) and HI₃; and passing the feed stream to one or more columns to provide a purified trifluoroacetyl iodide (TFAI) product stream.

The present disclosure also provides a method for removing iodine (I₂) from trifluoroacetyl iodide (TFAI), comprising adding a third component, such as trifluoroacetic acid, to a mixture of iodine (I₂) and trifluoroacetyl iodide (TFAI), wherein the third component is immiscible or nearly immiscible with iodine (I₂); heating the mixture to melt the iodine (I₂); allowing the mixture to settle into two layers; and separating the layers into a top and bottom layer; wherein the top layer comprises trifluoroacetyl iodide (TFAI) and the bottom layer comprises liquid iodine (I₂).

The above mentioned and other features of the disclosure, and the manner of attaining them, will become more apparent and will be better understood by reference to the following description.

### DESCRIPTION OF THE DRAWINGS

FIG. 1 shows a schematic for solvating and removing iodine (I₂) from trifluoroiodomethane (CF₃I).

FIG. 2 shows a schematic for an alternative method for solvating and removing iodine (I₂) from trifluoroiodomethane (CF₃I).

FIG. 3 shows a schematic for removing iodine (I₂) from trifluoroacetyl iodide (TFAI).

FIG. 4 shows a schematic for removing iodine (I₂) via phase separation as described in Example 7.

FIG. 5 shows the experimental setup described in Example 1.

FIG. 6 shows a GC/MS scan after iodine (I₂) and toluene were heated to 80° C. for 24 hours as described in Example 4.

FIG. 7 show a GC/MS scan after iodine (I₂) and toluene were heated to 250° C. for 2 weeks as described in Example 4.

### DETAILED DESCRIPTION

The present disclosure provides methods for removing iodine (I₂)-containing species from trifluoroacetyl iodide (TFAI) feedstock and from the reactor effluent stream during conversion of trifluoroacetyl iodide (TFAI) to trifluoroiodomethane (CF₃I).

Trifluoroiodomethane (CF₃I) may be produced according to the reactions shown below. In a first step, hydrogen iodide (HI) is produced according to Equation 1 below:

$${H}_{2} + {I}_{2} □2 HI \qquad Eq . 1$$

In one example of the process, the reactant stream reacts in the presence of a catalyst contained within a reactor to produce a product stream comprising hydrogen iodide according to Equation 1 above. The reactor may be a heated tube reactor, such as a fixed bed tubular reactor, including a tube containing the catalyst. The tube may be made of a metal such as stainless steel, nickel, and/or a nickel alloy, such as a nickel-chromium alloy, a nickel-molybdenum alloy, a nickel-chromium-molybdenum alloy, a nickel-iron-chromium alloy, or a nickel-copper alloy. The tube reactor may be heated, thus also heating the catalyst or the feed materials may be preheated before entering the reactor. Alternatively, the reactor may be any type of packed reactor.

The catalyst may be a nickel, cobalt, iron, nickel oxide, cobalt oxide, iron oxide, nickel iodide, cobalt iodide, and/or iron iodide catalyst on a support. Thus, the catalyst comprises at least one selected from the group of nickel, cobalt, iron, nickel oxide, nickel iodide, cobalt oxide, cobalt iodide, iron oxide, and iron iodide, wherein the catalyst is supported on a support. The support can be selected from the group of activated carbon, silica gel, zeolite, silicon carbide, metal oxides, and combinations thereof. Non-exclusive examples of the metal oxides include alumina, magnesium oxide, titanium oxide, zinc oxide, zirconia, chromia, and combinations thereof.

Prior to the reaction, the catalyst may be heated to a reaction temperature as of about 150° C. or greater, about 200° C. or greater, about 250° C. or greater, about 280° C. or greater, about 290° C. or greater, about 300° C. or greater, about 310° C. or greater, or about 320° C. or greater, about 330° C. or less, about 340° C. or less, about 350° C. or less, about 360° C. or less, about 380° C. or less, about 400° C. or less, about 450° C. or less, about 500° C. or less, about 550° C. or less, about 600° C. or less, or any value encompassed by these endpoints.

The reactant stream may be in contact with the catalyst for a contact time of about 0.1 second or longer, about 2 seconds or longer, about 4 seconds or longer, about 6 seconds or longer, about 8 seconds or longer, about 10 seconds or longer, about 15 seconds or longer, about 20 seconds or longer, about 25 seconds or longer, about 30 seconds or longer, about 40 seconds or shorter, about 50 seconds or shorter, about 60 seconds or shorter, about 70 seconds or shorter, about 80 seconds or shorter, about 100 seconds or shorter, about 120 seconds or shorter, or about 1,800 seconds or shorter.

Suitable operating pressures range may be about 0 psig or greater, about 10 psig or greater, about 20 psig or greater, about 50 psig or greater, about 75 psig or greater, about 100 psig or greater, about 120 psig or less, about 150 psig or less, about 250 psig or less, about 350 psig or less, about 450 psig or less, about 600 psig or less, or any value encompassed by these endpoints.

In the reactant stream, the H₂/I₂ mole ratio may be as low as about 1:1, about 1.5:1, about 2:1, about 2.5:1, about 2.7:1, or about 3:1, or as high as about 4:1, about 5:1, about 6:1, about 7:1, about 8:1, about 9:1, or about 10:1, or within any range defined between any two of the foregoing values.

In a second step, the hydrogen iodide (HI) is used to convert trifluoroacetyl chloride (TFAC) to trifluoroacetyl iodide (TFAI) according to Equation 2 below:

$$C {F}_{3} COCl + HI □ {CF}_{3} C O I + HCl \qquad Eq . 2$$

In one example of this process, the reaction may be conducted in a reactor, such as a heated tube reactor comprising a tube made of a metal such as carbon steel, stainless steel, nickel, and/or a nickel alloy, such as a nickel-chromium alloy, a nickel-molybdenum alloy, a nickel-chromium-molybdenum alloy, or a nickel-copper alloy. The reactor may be constructed of a metal lined with glass or polymers such as polytetrafluoroethylene (PTFE), perfluoroalkoxy alkanes (PFA), fluorinated ethylene propylene (FEP) and other fluoropolymers. The tube within the reactor may be heated or the feed materials may be preheated before entering the reactor. The reactor may be any type of packed bed reactor.

The hydrogen iodide and the trifluoroacetyl chloride in the reactant stream reacts in the presence of a catalyst contained within the reactor. The catalyst may comprise activated carbon, meso carbon, stainless steel, nickel, nickel-chromium alloy, nickel-chromium-molybdenum alloy, nickel-copper alloy, copper, alumina, platinum, palladium, or carbides, such as metal carbides, such as iron carbide, molybdenum carbide and nickel carbide, and non-metal carbides, such as silicon carbide, or combinations thereof.

The reactant stream may be in contact with the catalyst for a contact time of about 0.1 seconds or longer, about 0.5 seconds or longer, about 1 second or longer, about 2 seconds or longer, about 3 seconds or longer, about 5 seconds or longer, about 8 seconds or longer, about 10 seconds or longer, about 12 seconds or longer, or about 15 or longer, about 18 seconds or longer, about 20 seconds or shorter, about 25 seconds or shorter, about 30 seconds or shorter, about 35 seconds or shorter, about 40 seconds or shorter, about 50 seconds or shorter, about 60 seconds or shorter, about 80 seconds or shorter, or about 300 seconds shorter, or about 1800 seconds shorter, or any value encompassed by these endpoints.

The reaction temperatures may be about 0° C. or higher, about 25° C. or higher, about 35° C. or higher, about 40° C. or higher, about 50° C. or higher, about 60° C. or lower, about 90° C. or lower, about 120° C. or lower, about 150° C. or lower, about 200° C. or lower, about 250° C. or lower, or any value encompassed by these endpoints.

The pressure may be about 0 psig or higher, about 25 psig or higher, about 5 psig or higher, about 50 psig or higher, about 100 psig or higher, about 150 psig or higher, about 200 psig or higher, about 250 psig or higher, about 300 psig or lower, about 350 psig or lower, about 400 psig or lower, about 450 psig or lower, about 500 psig or lower, or any value encompassed by these endpoints.

The TFAC/HI mole ratio may be as low as about 1:10, about 1:5, about 1:2, about 1:1, about 2:1, about 3:1, about 4:1, or as high as about 5:1, about 6:1, about 7:1, about 8:1, about 9:1, or about 10:1, or within any range defined between any two of the foregoing values. Preferably, the TFAC/HI mole ratio is from 1:2 to 2:1. More preferably, the TFAC/HI mole ratio is from 1:1 to 2:1.

Finally, in a third step as disclosed in U.S. patent application Ser. No. 16/549,412, trifluoroiodomethane (CF₃I) can be formed via the decomposition of trifluoroacetyl iodide (TFAI) according to Equation 3 below:

$$C {F}_{3} C O I □ C {F}_{3} I + C O \qquad Eq . 3$$

The reaction may take place in a heated tube reactor or an electric heater reactor. The electric heater reactor may be an impedance tube reactor with the electrical current passing directly through the heater tube wall utilizing alternating current at low voltage. Alternatively, the electric heater reactor may be an immersion-type electric heater. This novel immersion-type electric heater may be a system using electricity as the heating medium, with the reaction occurring on the outside of the heating elements. The reactor may comprise a metal alloy encasing Nichrome heating elements within compacted magnesium oxide (MgO) powder.

The reactor may comprise a metal alloy, such as Inconel® 600, Inconel® 625, Incoloy® 800 and Incoloy® 825, for example.

The heater surface may be a catalytic surface or a non-catalytic surface. Suitable metal surfaces may include electroless nickel, nickel, stainless steel, nickel-copper alloy, nickel-chromium-iron alloy, nickel-chromium alloy, nickel-chromium-molybdenum alloy, or combinations thereof.

The reaction may take place in a heated tube reactor comprising a tube made of a metal such as stainless steel, nickel, and/or a nickel alloy, such as a nickel-chromium alloy, a nickel-molybdenum alloy, a nickel-chromium-molybdenum alloy, or a nickel-copper alloy. The tube within the reactor may be heated. The reactor may also include any type of packed bed reactor. The packing may be a catalyst or an inert material that improves heat transfer and promotes mixing of the reactants and products.

The reaction may be carried out at a temperature of about 200° C. or greater, about 250° C. or greater, about 300° C. or greater, about 350° C. or greater, about 400° C. or lower, about 450° C. or lower, about 500° C. or lower, about 550° C. or lower, about 600° C. or lower, or within any range encompassing these endpoints. Preferably, the reaction may be carried out at a temperature from about 300° C. to about 500° C. More preferably, the reaction may be carried out at temperature from about 300° C. to about 400° C.

The reaction may be carried out at a pressure of about 0 psig or greater, about 5 psig or greater, about 20 psig or greater, about 50 psig or greater, about 70 psig or greater, about 100 psig or greater, about 150 psig or lower, about 200 psig or lower, about 225 psig or lower, about 250 psig or lower, about 275 psig or lower, about 300 psig, or within any range encompassing these endpoints.

The contact time of the reaction may be about 0.1 second or greater, about 1 second or greater, about 5 seconds or greater, about 10 seconds or greater, about 60 seconds or greater, about 100 seconds or less, about 150 seconds or less, about 200 seconds or less, about 250 seconds or less, about 300 seconds or less, about 600 seconds or less, or within any range encompassing these endpoints.

The reaction may be conducted in the presence of a catalyst. The catalyst may comprise stainless steel, nickel, nickel-chromium alloy, nickel-chromium-molybdenum alloy, nickel-copper alloy, copper, alumina, silicon carbide, platinum, palladium, rhenium, activated carbon, such as such as Norit PK 3-5, Calgon or Shirasagi carbon, or combinations thereof. Alternatively, the reaction may be conducted in the absence of a catalyst.

The presence of iodine (I₂)-containing species in the trifluoroacetyl iodide (TFAI) may be disadvantageous from the standpoints of reduced productivity of the desired product and increased operational cost. Iodine (I₂)-containing species may include one or more of iodine (I₂), HI₃, or combinations thereof.

The presence of iodine (I₂)-containing species, such as I₂ and HI₃, together with additional I₂ formed during the reaction may cause increased corrosion of equipment and/or operational difficulties including flow, pressure control and plugging issues. Additionally, the presence of these iodinated species may increase the formation of unwanted by-products, such as trifluoromethane and iodine (I₂), which may form during the conversion step of trifluoroacetyl iodide (TFAI) to trifluoroiodomethane (CF₃I) due to the presence of hydrogen-containing species, such as HI and HI₃, thereby lowering the overall process yield and possibly causing difficulties in purification of the trifluoroiodomethane (CF₃I) final product.

The present disclosure provides a method wherein at least a solvent is used to prevent the formation of slid iodine (I₂). The present disclosure further provides a method wherein at least one column is used to remove iodine (I₂)-containing species. This column may be positioned such that iodine (I₂)-containing species, such as HI₃ and I₂, may be removed from the trifluoroiodomethane (CF₃I) product stream or from the trifluoroacetyl iodide (TFAI) raw material stream, as shown in Equation 3 above. For example, a solvent may be added to the reactor effluent in order to prevent the formation of solid iodine (I₂) by solvation of iodine (I₂). Without being bound by theory, limiting the formation iodine (I₂) solids may cause operational issues, such as plugging and corrosion as well as undesired byproducts formation.

Suitable solvents are those with high iodine (I₂) solubility, such as benzene and alkyl-substituted benzenes. Solvents may include benzene, toluene, xylenes, mesitylene (1,3,5-trimethylbenzene), ethyl benzene and the like; dimethylformamide (DMF), dimethyl sulfoxide, (DMSO), and ionic liquids such as imidazolium salts and caprolactamium hydrogen sulfate, for example, and combinations thereof.

Toluene, for example, may be a suitable solvent due to high iodine (I₂) solubility and low toxicity. Further advantages may include the lack of reactivity of toluene towards iodine (I₂), HI₃ as well as other components in the system and the ease of separation of toluene from trifluoroacetyl iodine (TFAI).

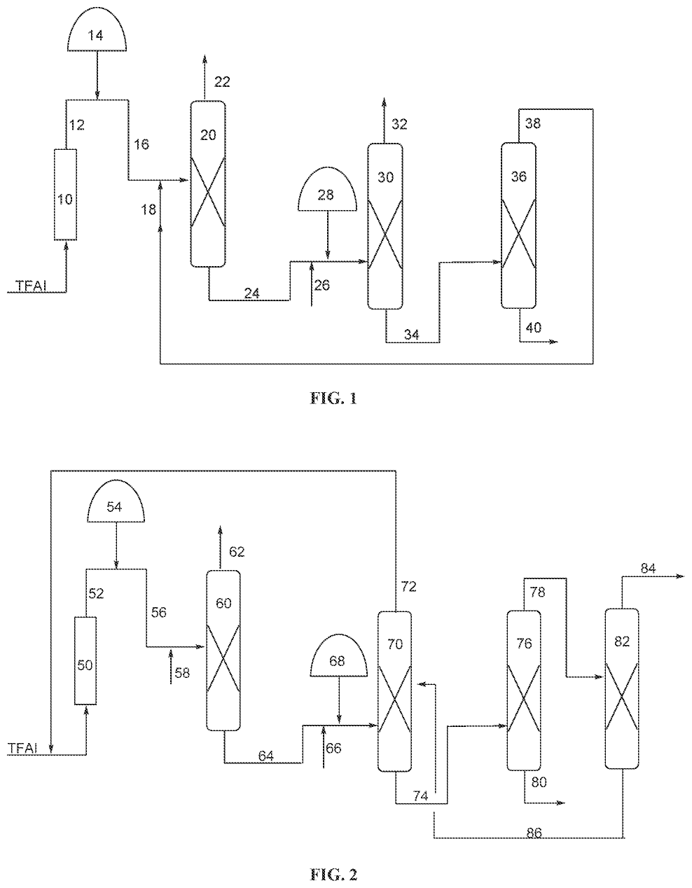

Iodine (I₂)-containing species may be removed from the trifluoroiodomethane (CF₃I) product stream using the method shown in FIG. 1. A feed stream comprising trifluoroacetyl iodide (TFAI) is passed through a reactor 10 to provide a product stream 12 comprising trifluoroiodomethane (CF₃I), trifluoroacetyl iodide (TFAI), carbon monoxide (CO), and at least an iodine (I₂)-containing species selected from HI₃, and iodine (I₂). A solvent such as toluene 14 may be added to reactor 10 outlet line to prevent the formation of solid iodine in the stream 16. Optionally, a stream 18 derived from the previous step of the reaction, comprising crude trifluoroacetyl iodide (TFAI), and at least an iodine (I₂)-containing species selected from HI₃, and iodine (I₂), may be combined with stream 16 before said stream is conveyed to a first column 20. A first overhead product 22 from the first column 20 comprises trifluoroiodomethane (CF₃I), carbon monoxide (CO), and small amounts of low-boiling impurities. The first overhead product 22 may be further processed to provide refrigerant grade trifluoroiodomethane (CF₃I). A first bottoms product 24 comprises solvent, unreacted trifluoroacetyl iodide (TFAI), at least an iodine (I₂)-containing species selected from HI₃, and iodine (I₂), and high-boiling components. Optionally, stream 26 comprising a solvent such as toluene, unreacted trifluoroacetyl iodide (TFAI), and at least an iodine (I₂)-containing species selected from HI₃, and iodine (I₂) may be combined with stream 24. Additional solvent such as toluene 28 may be added to the stream before it is conveyed to a second column 30. A second overhead product 32 of second column 30 may comprise purified trifluoroacetyl iodide (TFAI). A second bottoms product 34 of second column 30 may comprise a solvent such as toluene and at least an iodine (I₂)-containing species selected from HI₃, and iodine (I₂). The solvent prevents solid iodine (I₂) from being deposited as the stream 34 is conveyed to a third column 36. A third overhead product 38 of third column 36, comprising a solvent, such as toluene, may be recycled back to reactor 10 outlet line, to the second column 30, or combined with stream 18. A third bottoms product 40 comprises purified iodine (I₂) and solvent. The purified iodine (I₂) may be recycled back as a feed stream for the reaction depicted in Equation 1, or may be stored for an alternate use.

The processes of the present disclosure, such as the example described above, may be run as a continuous process or may be conducted as an intermittent process.

The columns may be operated in a manner such that the overhead temperature is different than the bottom temperature. The reboiler temperatures described below refer to the bottom temperatures of the columns.

The reboiler temperatures of the first two columns may generally be maintained at a temperature below about 150° C., such as about 150° C. or less, about 140° C. or less, about 130° C. or less, about 120° C. or less, about 110° C. or less, or about 100° C. or less.

The reboiler temperature of the third column may be maintained at a temperature below about 250° C., such as about 250° C. or less, about 240° C. or less, about 230° C. or less, about 220° C. or less, about 210° C. or less, about 200° C. or less, about 190° C. or less, about 180° C. or less, or about 170° C. or less.

Pressures in the columns is not critical. The three columns may be operated at a pressure of about 0 psig or higher, about 10 psig or higher, about 25 psig or higher, about 50 psig or higher, about 75 psig or higher, about 100 psig or higher, about 125 psig or higher, about 150 psig or higher, about 175 psig or less, about 200 psig or less, about 225 psig or less, about 250 psig or less, about 275 psig or less, about 300 psig or less, or any value encompassed by these endpoints.

Alternatively, iodine (I₂)-containing species may be removed from the trifluoroiodomethane (CF₃I) product stream using the method shown in FIG. 2. A feed stream comprising trifluoroacetyl iodide (TFAI) is passed through a reactor 50 to provide a product stream 52 comprising trifluoroiodomethane (CF₃I), trifluoroacetyl iodide (TFAI), carbon monoxide (CO), and at least an iodine (I₂)-containing species selected from HI₃, and iodine (I₂). A solvent such as toluene 54 may be added to reactor 50 outlet line to prevent the formation of solid iodine in the stream 56. Optionally, a stream 58 derived from the previous step of the reaction, comprising crude trifluoroacetyl iodide (TFAI), and at least an iodine (I₂)-containing species selected from HI₃, and iodine (I₂), may be combined with stream 56 before said stream is conveyed to a first column 60. A first overhead product 62 from the first column 60 comprises trifluoroiodomethane (CF₃I), carbon monoxide (CO), and small amounts of low-boiling impurities. The first overhead product 62 may be further processed to provide refrigerant grade trifluoroiodomethane (CF₃I). A first bottoms product 64 comprises solvent, unreacted trifluoroacetyl iodide (TFAI), trifluoroacetic acid (TFA), at least an iodine (I₂)-containing species selected from HI₃, and iodine (I₂), and high-boiling components. Stream 66 derived from the previous step of the reaction, comprising crude trifluoroacetyl iodide (TFAI), and at least an iodine (I₂)-containing species selected from HI₃, and iodine (I₂), may be combined with stream 64. Additional solvent such as toluene 68 may be added to the stream before it is conveyed to a second column 70. A second overhead product 72 of second column 70 may comprise purified trifluoroacetyl iodide (TFAI), which may be recycled back to the reactor 50. A second bottoms product 74 of second column 70 may comprise a solvent such as toluene, trifluoroacetic acid (TFA), high-boiling impurities, and at least an iodine (I₂)-containing species selected from HI₃, and iodine (I₂). The solvent prevents solid iodine (I₂) from being deposited as the stream 74 is conveyed to a third column 76. A third overhead product 78 of third column 76, comprising a solvent, such as toluene, trifluoroacetic acid (TFA), and high boiling impurities may be conveyed to a fourth column 82, while a third bottoms product 80, comprising purified iodine (I₂), HI₃ and trace solvent may be collected. The fourth overhead product 84 from the fourth column 80 may be purged, while the fourth bottoms product 86 comprising a solvent such as toluene from the fourth column 82 may be recycled back to the second column 70.

In the processes described above, the purified trifluoroacetyl iodide (TFAI) may be essentially free of iodine (I₂). As used herein, the phrase “essentially free of iodine (I₂)” is defined as a concentration of iodine of less than 2000 ppm, preferably less than 1000 ppm, more preferably less than 500 ppm, and most preferably less than 200 ppm.

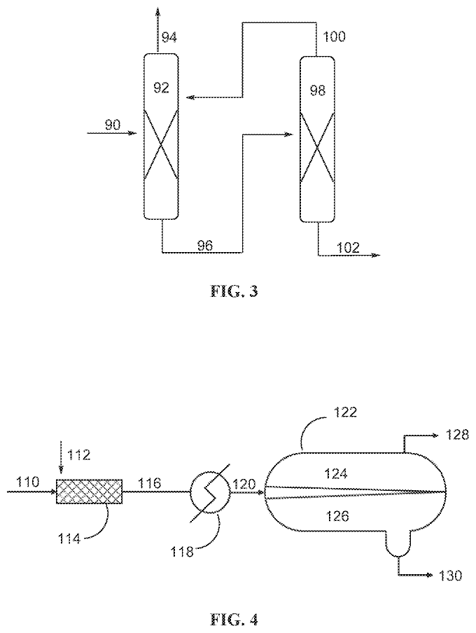

Another alternative for removing iodine (I₂)-containing species such as I₂, and HI₃ from a feed stream using vapor/liquid contacting columns is shown in FIG. 3. In this process, iodine (I₂) may be recovered as a liquid, which provides an advantage over recovery of solid iodine as it does not need to be melted off of any equipment and does not cause issues such as plugging.

As shown in FIG. 3, a feed stream 90 comprising the components to be recovered, such as trifluoroacetyl iodide (TFAI), and trifluoroacetic acid, for example, is fed to a first column 92, along with a solvent. The first column 92 includes a condenser and rectification section to allow for reflux. Optionally, the first column 92 includes a reboiler and stripping section. A first overhead vapor product 94 contains the component to be recovered, such as trifluoroacetyl iodide (TFAI). A first bottoms product 96 may include a solvent and iodine (I₂). The first bottoms product 96 is conveyed to a second column 98. The second column 98 includes a reboiler and a stripping section. Optionally, the second column 98 includes a condenser and a rectification section. A second overhead product 100 may include solvent in the form of a vapor or a liquid. The overhead product 100 may be recycled back to the first column 92. Optionally, fresh solvent may be added to stream 100 (not shown). A second bottoms product 102 from the second column 98 may include liquid iodine (I₂).

Optionally, further columns may be included to conduct partial separations.

The solvent in the method described above may be a solvent with high solubility of iodine (I₂). The solvent may have a vapor pressure higher than that of iodine (I₂) but lower than that of the components being recovered in the gas stream. Suitable solvents may include benzene; xylenes, such as paraxylene, metaxylene, and alkylated benzenes, such as mesitylene (1,3,5-trimethylbenzene) and toluene; dimethylformamide (DMF); and dimethyl sulfoxide (DMSO), for example.

The solvent type, solvent circulation rate, first column pressure, and first column reboiler heat input are selected such that the iodine (I₂) does not form a solid phase. For example, the temperature may be above 114° C., the melting point of iodine (I₂). This permits the first overhead product to be substantially free of iodine (I₂) as the iodine is dissolved in the solvent and exists the first column in the bottom product.

The solvent type, solvent circulation rate, second column pressure, and second column reboiler heat input are selected such that the iodine (I₂) does not form a solid phase. For example, the temperature may be above 114° C., the melting point of iodine (I₂). This permits the second overhead product to be substantially free of iodine (I₂) as the iodine is present as a liquid and exits the second column as the bottom product. The operating pressure of the second column may be lower than that of the first column.

As yet another alternative, removing iodine (I₂) from a liquid stream using extraction and phase separation is shown in FIG. 4. Liquid iodine (I₂) may be recovered from a feed stream by the addition of a separate component, such as a solvent, followed by phase separation. Suitable components may be compatible with the reaction and recovery process, may be miscible with the organic components present, and may be substantially immiscible with iodine (I₂). One such component is trifluoroacetic acid (TFA).

As shown in FIG. 4, a feed stream 110 comprising the components to be recovered, such as trifluoroacetyl iodide (TFAI), and trifluoroacetic acid, for example, is combined with another feed stream 112 which is substantially TFA and fed through a mixing device 114. The combined stream 116 is conveyed to a heater 118 and heated to a temperature above the melting point of iodine. The heated mixture 120 is fed to a phase separator and fed to phase separator 122 to allow the organic layer 124 comprising TFAI, TFAC and TFA and the iodine (I₂) layer 126 to separate into two liquid layers. Stream 128, from the top organic layer may be decanted to recover the desired products. Preferably, TFA is separated by distillation or series of distillation steps for recycle via stream 112. Stream 130, from the bottom layer, comprises liquid iodine (I₂) that is essentially free of solid iodine (I₂), which may then be recycled back as a feed stream for the reaction depicted in Equation 1, or may be stored for an alternate use.

The temperature may be about 114° C. or higher, about 115° C. or higher, about 120° C. or higher, about 125° C. or lower, about 130° C. or lower, about 135° C. or lower, about 140° C. or lower, or any value encompassed by these endpoints.

In the processes described above, the component to be recovered such as trifluoroacetyl iodide (TFAI) may be essentially free of iodine (I₂). As used herein, the phrase “essentially free of iodine (I₂)” is defined as a concentration of iodine of less than 2000 ppm, preferably less than 1000 ppm, more preferably less than 500 ppm, and most preferably less than 200 ppm.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” is also considered as disclosing the range defined by the absolute values of the two endpoints.

The following non-limiting Examples serve to illustrate the disclosure.

### EXAMPLES

### Example 1: Iodine (I₂) Absorption into Toluene

Six studies on iodine (I₂) absorption using toluene as a solvent were performed. Each study was conducted after reaching steady-state reactor conditions during different continuous runs for the decomposition of trifluoroacetyl iodide (TFAI) to CF₃I and CO, during which the average conversion of trifluoroacetyl iodide (TFAI) was about 65% and the reaction pressure was 25 psig.

In each case, the trifluoroacetyl iodide (TFAI) reactor effluent stream was directed to a collection system consisting of a 950 ml high pressure Fisher-Porter (F-P) Tube containing toluene (i.e., a toluene bubbler), followed by a second 950 ml high pressure F-P Tube dry ice trap. In each experiment, the 950 ml toluene bubbler was initially charged with 300 grams of toluene and heated to 45-50° C. by wrapping electrical heat tape loosely around the bubbler. The 950 ml dry ice trap was placed in a Dewar containing an acetone/dry ice slush at −81° C. The reactor effluent was fed to the 950 ml F-P tube toluene bubbler through a dip tube. The toluene then absorbed the majority of the iodine (I₂), and the majority of the trifluoroacetyl iodide (TFAI) was condensed.

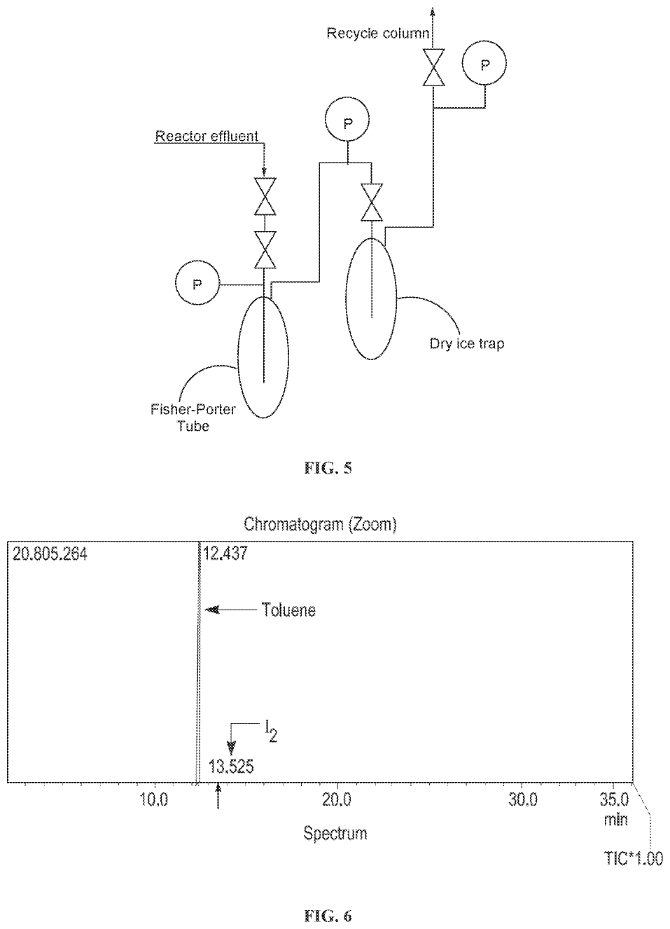

The stream exiting the top of the bubbler was substantially free of iodine (I₂). The iodine concentration may be determined by titration. An example of this method is to add a known amount of sample to 36 grams of deionized water, mixing, adding 4.0 grams of potassium iodide (KI), and titrating the mixture with sodium thiosulfate. This essentially iodine (I₂)-free stream was fed to the second 950 ml F-P tube dry ice trap in which trifluoroiodomethane (CF₃I), residual unreacted trifluoroacetyl iodide (TFAI), and by-products were collected (including entrained or volatized toluene). The exit stream from the dry ice trap was then directed to the trifluoroiodomethane (CF₃I) crude column for about 16 hours to allow the carbon monoxide (CO) produced in the course of the reaction to vent and avoid pressure build-up in excess of 25 psig in the experimental apparatus. FIG. 5 shows an experimental set up for these reactions.

The initial and final weight of the trifluoroacetyl iodide (TFAI) feed cylinder was recorded and the total amount of material collected in the bubbler and dry ice trap was recorded. The toluene and additional material remaining in the bubbler, as well as the material collected in the dry ice traps, were sampled and the iodine (I₂) concentration of each was determined by titration. Data for each of the experiments is shown below in Table 1, in which the net weight of the bubbler includes the 300 g of toluene that was charged at the beginning of the run. As shown in Table 1, the toluene bubbler absorbed between 93.85% and 98.45% of the iodine (I₂) in the reactor effluent stream.

The samples were also analyzed by gas chromatography/mass spectrometry (GC/MS) which verified that very little reaction between the toluene and reactor effluent material had occurred (only single digit ppm levels of by-products attributed to the possible reaction with toluene were found).

TABLE 1

| | | Bubbler | | | Dry ice trap | | | | |
|---|---|---|---|---|---|---|---|---|---|
| Run | TFAI | Contents | Conc. | I₂ | Contents | Conc. | I₂ | Total | % I₂ in |
| # | fed, g | Wt., g | I₂, ppm | Captured, g | Wt., g | I₂, ppm | Captured, g | I₂, g | bubbler |
| 1 | 1907 | 563 | 12960 | 7.3 | 1473 | 78 | 0.11 | 7141 | 98.45 |
| 2 | 1816 | 629 | 14394 | 9.0 | 1318 | 130 | 0.17 | 9.17 | 98.13 |
| 3 | 2043 | 684 | 6143 | 4.2 | 1488 | 67 | 0.10 | 4.30 | 97.68 |
| 4 | 1861 | 681 | 6150 | 4.2 | 1310 | 92 | 0.12 | 4.32 | 97.20 |
| 5 | 1861 | 592 | 11299 | 6.7 | 1389 | 98 | 0.14 | 6.84 | 98.01 |
| 6 | 1861 | 529 | 10510 | 5.6 | 1402 | 260 | 0.36 | 5.96 | 93.85 |

### Example 2: Toluene Recovery

The toluene was recovered from material collected from a toluene bubbler in an experiment similar to those described in Example 1. The recovery experiment was conducted in a 20-stage Oldershaw glass distillation column equipped with a magnetic splitter. The toluene bubbler material (526 g) was charged to a 1 L glass round bottom flask reboiler. The reboiler was placed in an electric heating mantle and gently heated to drive off non-condensables and ‘lights’. Reflux was observed when the head temperature was between 25° C. and 29° C. The material collected in the product receiver through the vapor line at this temperature was designated as the Lights Cut. The material was pink, was estimated to have a volume roughly 30 ml, and was not further quantified.

When the head temperature reached 30° C. and there was strong reflux, a distillate cut was taken with a splitter timer of 10 seconds reflux to 1 second take-off and, was designated as Main Cut #1. All of the material collected in the distillate receiver at a head temperature between 30° C. and 35° C. A total of 157.3 grams of pink liquid was collected. The gas chromatography (GC) analysis showed that the material consisted of 98.3% trifluoroacetyl iodide (TFAI) with trifluoroiodomethane (CF₃I), trifluoroacetic acid (TFA), and toluene also present in minor amounts. The concentration of iodine (I₂) was determined by titration was 378 ppm. This result showed that the trifluoroacetyl iodide (TFAI) can be successfully recovered from the toluene while leaving the majority of the dissolved I₂ behind.

Next, an intermediate cut of material was taken from that collected at a head temperature between 35° C. and 110° C. using the same splitter setting. Dark purple material (28.1 g) was collected in the distillate receiver. The GC analysis showed that the material consisted of 99.6% toluene including some trifluoroacetyl iodide (TFAI) and trifluoroacetic acid (TFA). The iodine (I₂) concentration was not determined.

When the head temperature was steady at 110° C. with strong reflux, a second main distillate cut was collected and designated as Main Cut #2.The same splitter setting was used. The cut was collected over about 12 hours, until the reboiler temperature reached 117° C. A total of 115.1 grams of orange liquid was collected. The reboiler temperature during this cut was between 112.5° C. and 117° C., and the head temperature ranged from 110° C. to 110.5° C. GC analysis showed the material consisted of greater than 99.9% toluene. The iodine (I₂) concentration as determined by titration was 564 ppm. This result showed that toluene can be successfully recovered from the toluene/I₂ mixture while leaving the majority of the dissolved iodide (I₂) behind.

The reboiler residue was drained from the flask and amounted to 94.5 g. GC analysis was not performed on the material, but it was analyzed for iodine (I₂) concentration by titration, which showed 11,049 ppm iodine (I₂). Table 2 shows the pertinent data for the toluene recovery experiment.

TABLE 2

| | | Head | | | | |
|---|---|---|---|---|---|---|
| Sample | Amount | Temp | GC % | I₂ | I₂ | |
| description | (g) | (° C.) | area | (ppm) | (g) | Notes |
| Starting | 526 | | N/A | >2000 | — | Bubbler material is |
| material | | | | | | dark purple |
| Lights cut | 50 (est.) | <30 | N/A | N/A | N/A | Pink material |
| Main Cut 1 | 157.3 | 30-35 | 98.3 TFAI, | 378 | 0.0595 | Pink material |
| | | | 0.98 CF₃I, | | | |
| | | | 0.15 TFA, | | | |
| | | | 0.48 | | | |
| | | | toluene, | | | |
| | | | 0.09 other | | | |
| Intermediate | 28.1 | 35-110 | 99.63 | N/A | N/A | Purple material; |
| Cut | | | toluene, | | | column bumped |
| | | | 0.16 TFAI, | | | during collection |
| | | | 0.0292 TFA, | | | |
| | | | 0.18 others | | | |
| Main Cut 2 | 115.1 | 110-110.5 | >99.9% | 564 | 0.0649 | Orange material |
| | | | toluene | | | |
| Reboiler | 94.5 | — | Not | 11049 | 1.0441 | Dark purple |
| Residue | | | determined | | | material |

### Example 3: Toluene Recovery

A second experiment was conducted to recover the toluene from the material collected from one of the toluene bubblers used in Example 1. The same batch glass distillation apparatus was used as in Example 2. The toluene bubbler material collected in Run 2 of Example 1 (558.1 g) was charged to the toluene recovery apparatus consisting of a 1 L glass round bottom flask reboiler. The iodine (I₂) concentration was 14,304 ppm. The reboiler was placed in an electric heating mantle and gently heated to drive off non-condensables and ‘lights’. After a short time, reflux was observed at a head temperature of 25° C. to 29° C. A very small amount of material (0.9 grams) was collected in the product receiver through the vapor line and was designated as the “Lights Cut”. An attempt to transfer the lights cut to a cylinder was unsuccessful; therefore, no analysis was performed on this cut.

When the head temperature reached 30° C. with strong reflux, a distillate cut was taken with a splitter timer of 10 seconds reflux to 1 second take-off and was designated as Main Cut #1. Material was collected in the distillate receiver at a head temperature range of 30° C. to 32° C. A total of 208.4 grams of liquid with a slight pink color was collected. The iodine (I₂) concentration as determined by titration was 153 ppm. A gas chromatography (GC) analysis showed 97.17% trifluoroacetyl iodide (TFAI), 1.38% toluene, 1.12% trifluoroiodomethane (CF₃I), and 0.19% trifluoroacetic acid (TFA).

Next, an intermediate cut of material was collected with the same splitter setting at a head temperature of 32° C. to 110° C. The coral-colored liquid (57.3 g) was collected in the distillate receiver. The iodine (I₂) concentration as determined by titration was 451 ppm. A GC analysis showed 99.61% toluene, 0.23% trifluoroacetyl iodide (TFAI), and 0.11% trifluoroacetic acid (TFA).

When the head temperature was steady at 110° C. with strong reflux, a second main distillate cut was collected and designated as Main Cut #2. The same splitter setting was used. During this cut, the head temperature ranged from 110° C. to 110.5° C., and the reboiler temperature ranged from 112.5° C. to 200° C. A light pink-colored liquid (196.1 g) was collected in the distillate receiver. The iodine (I₂) concentration as determined by titration was 202 ppm. GC analysis showed 99.996% toluene. These results showed that toluene can be successfully recovered from the toluene/I₂ mixture while leaving the majority of the dissolved iodine (I₂) behind.

GC analysis of the dark purple residue in the reboiler was attempted. However, the concentration of iodine (I₂) was high enough that solid crystals could be visually observed in the reboiler, and further addition of toluene was not sufficient to remove them all. Due to the large amount of iodine (I₂) present, GC analysis could not be completed.

Table 3 shows the data for this toluene recovery experiment.

TABLE 3

| | | Head | | I2 | | |
|---|---|---|---|---|---|---|
| Sample | Amount | T | GC | conc. | I2 | |
| Description | (g) | (° C.) | % area | (ppm) | (g) | Notes |
| Starting | 558.1 | — | N/A | 14,304 | 7.9831 | Dark purple |
| material | | | | | | material |
| Lights Cut | 0.9 | <30 | N/A | N/A | N/A | Insignificant |
| | | | | | | amount |
| | | | | | | collected |
| Main Cut 1 | 208.4 | 30-32 | 97.17 TFAI, | 153 | 0.0319 | Light pink |
| | | | 1.38 toluene, | | | material |
| | | | 1.12 CF₃I, | | | |
| | | | 0.19 TFA | | | |
| Intermediate | 57.3 | 32-110 | 99.61 | 451 | 0.0258 | Coral-colored |
| Cut | | | toluene, | | | material |
| | | | 0.23 TFAI, | | | |
| | | | 0.11 TFA | | | |
| Main Cut 2 | 196.1 | 110-110.5 | 99.996 | 202 | 0.0396 | Light pink |
| | | | toluene | | | material |
| Reboiler | 54.03 | — | N/A | 57,490 | 3.1062 | Deep purple |
| residue | | | | | | material |
| Mass balance | 92.6 | | | | | |

### Example 4: Reactivity of Toluene and Iodine (I₂) at 80° C. and 250° C.

A mixture of 10 wt. % iodine (I₂) and 90 wt. % toluene was charged to a 600 ml autoclave, mixed, and heated to 80° C. for 24 hours. The liquid was then sampled via a dip tube, and analyzed by ¹H NMR and gas chromatography/mass spectrometry (GC/MS) to determine whether any iodine-containing compounds were present.

In a second run, a mixture of 10 wt. % iodine (I₂) and 90 wt. % toluene was again charged to a 600 ml autoclave, mixed, and heated to 250° C. for 2 weeks, after which no increase in pressure was observed. The liquid was then sampled via a dip tube and analyzed by ¹H NMR and GC/MS to see whether any iodine-containing compounds were present.

The experimental data can be found in Table 4 below.

TABLE 4
Iodine and toluene thermal stability

| I₂ (wt. %) | Toluene (wt. %) | Temp. and duration | Result |
|---|---|---|---|
| 10 | 90 | 80° C., 24 hr | NR |
| 10 | 90 | 250° C., 2 weeks | NR |

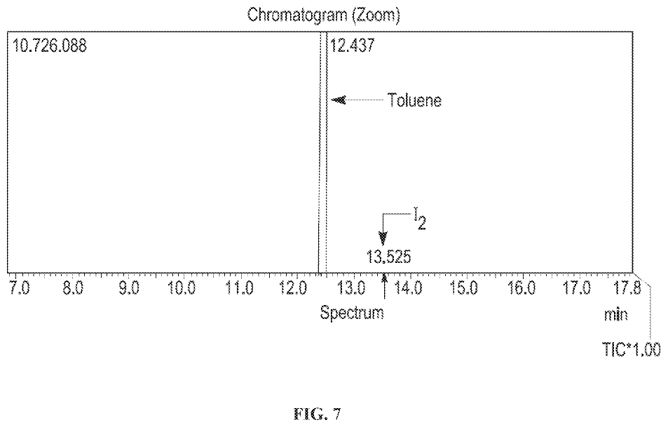

Based on 1H NMR and GC/MS, it was concluded that no reaction (NR) occurs between toluene and iodine (I₂) at 80° C. after 24 hours, nor at 250° C. after 2 weeks. Although no iodinated toluene species or other iodine-containing species were detected by GC/MS after 2 weeks at 250° C., small amounts (<0.5%) of benzene, xylene, p-xylene were detected. The GC/MS scans for both runs are shown in FIGS. 6 and 7.

### Example 5: Toluene and Iodine (I₂) Recovery from Trifluoroiodomethane (CF₃I)

Trifluoroacetyl iodide (TFAI) is passed through a reactor to provide a product stream comprising trifluoroiodomethane (CF₃I), trifluoroacetyl iodide (TFAI), carbon monoxide (CO), hydrogen iodide (HI), HI₃, and iodine (I₂). Toluene is added to prevent the formation of solid iodine, and the stream is conveyed to a first column. A first overhead product comprising trifluoroiodomethane (CF₃I), carbon monoxide (CO), and small amounts of low-boiling impurities is collected. A first bottoms product comprising unreacted trifluoroacetyl iodide (TFAI), iodine (I₂), HI₃, and high-boiling components is conveyed to a second column. A second overhead product comprising trifluoroacetyl iodide (TFAI) with less than 200 ppm iodine (I₂) is collected. A second bottoms product comprising toluene and iodine (I₂) is conveyed to a third column. A third overhead product comprising toluene is recycled back to the second column, and a third bottoms product comprising iodine (I₂) and solvent is collected.

### Example 6: Iodine (I₂) Recovery from Trifluoroacetyl Iodide (TFAI)

A feed stream comprising trifluoroacetyl iodide (TFAI), a solvent, and over 2000 ppm iodine (I₂) is conveyed to a first column equipped with a condenser and rectification section to allow for reflux. A first overhead vapor product comprises trifluoroacetyl iodide (TFAI) with less than 200 ppm iodine (I₂). A first bottoms product comprising the solvent and iodine (I₂) is conveyed to a second column, which is equipped with a reboiler and a stripping section. A second overhead product comprises the solvent, and is recycled back to the first column. A second bottoms product from the second column comprises liquid iodine (I₂).

### Example 7: Iodine (I₂) Recovery from Trifluoroacetic Acid (TFA)

A mixture comprising TFAI and over 2000 ppm iodine (I₂) is mixed with trifluoroacetic acid (TFA) and heated to 130° C. and allowed to settle to form two immiscible layers. The mixture is allowed to cool to room temperature. The top organic layer with less than 200 ppm iodine (I₂) is decanted. The apparatus is reheated to remelt the iodine layer to collect a bottom layer comprising liquid iodine (I₂). A schematic of the experimental set up is shown in FIG. 4.

### ASPECTS

Aspect 1 is a method for solvation and removal of an iodine (I₂)-containing species comprising the following steps: providing a feedstock comprising trifluoroacetyl iodide (TFAI) and the iodine (I₂)-containing species; adding a solvent to the feedstock stream to provide a mixture comprising the solvent, trifluoroacetyl iodide (TFAI) and the iodine (I₂)-containing species; and passing the mixture to one or more columns to obtain a purified stream comprising trifluoroacetyl iodide (TFAI).

Aspect 2 is the method of Aspect 1, wherein the solvent comprises one or more of benzene, toluene, xylenes, mesitylene (1,3,5-trimethylbenzene), ethyl benzene, alkylated benzenes, dimethylformamide (DMF), dimethyl sulfoxide, (DMSO), imidazolium salts and caprolactamium hydrogen sulfate.

Aspect 3 is the method of Aspect 2, wherein the solvent comprises toluene.

Aspect 4 is the method of any one of Aspects 1-3, further comprising: passing the mixture to a first column to provide a first overhead product and a first bottoms product comprising the solvent, trifluoroacetyl iodide (TFAI), and at least an iodine (I₂)-containing species selected from HI₃ and iodine (I₂); passing the first bottoms product to a second column to provide a second overhead product comprising purified trifluoroacetyl iodide (TFAI) and a second bottoms product comprising the solvent and at least an iodine (I₂)-containing species selected from HI₃ and iodine (I₂); and passing the second bottoms product to a third column to provide a third overhead comprising the solvent and a third bottoms product comprising iodine (I₂) and the solvent.

Aspect 5 is the method of Aspect 4, wherein the purified trifluoroacetyl (TFAI) contains less than 2000 ppm iodine (I₂).

Aspect 6 is the method of any one of Aspects 1-5, further comprising recycling the solvent.

Aspect 7 is the method of any one of Aspects 1-6, further comprising: passing the mixture first column to provide a first overhead product and a first bottoms product comprising the solvent, trifluoroacetyl iodide (TFAI), trifluoroacetic acid (TFA), and at least an iodine (I₂)-containing species selected from HI₃ and iodine (I₂); passing the first bottoms product to a second column to provide a second overhead product comprising trifluoroacetyl iodide (TFAI) and a second bottoms product comprising the solvent, trifluoroacetic acid (TFA), high-boiling impurities, and at least an iodine (I₂)-containing species selected from HI₃ and iodine (I₂); passing the second bottoms product a third column to provide a third overhead product comprising the solvent, trifluoroacetic acid (TFA), and high boiling impurities, and a third bottoms product comprising iodine (I₂); passing the third overhead product to a fourth column to provide a fourth overhead product and a fourth bottoms product; purging the fourth overhead product; and recycling the fourth bottoms product back to the second column.

Aspect 8 is the method of any one of Aspects 1-7, wherein the purified trifluoroacetyl (TFAI) contains less than 2000 ppm iodine (I₂).

Aspect 9 is the method of any one of Aspects 1-8, further comprising recycling the solvent.

Aspect 10 is a method of removing iodine (I₂) from a stream comprising trifluoroacetyl iodide (TFAI), and at least an iodine (I₂)-containing species selected from iodine (I₂) and HI₃, the method comprising: providing a feed stream, a solvent, and at least an iodine (I₂)-containing species selected from iodine (I₂) and HI₃; and passing the feed stream to one or more columns to provide a purified trifluoroacetyl iodide (TFAI) product stream.

Aspect 11 is the method of Aspect 10, wherein the solvent comprises one or more of benzene, toluene, xylenes, mesitylene (1,3,5-trimethylbenzene), ethyl benzene, alkylated benzenes, dimethylformamide (DMF), dimethyl sulfoxide, (DMSO), imidazolium salts and caprolactamium hydrogen sulfate.

Aspect 12 is the method of either Aspect 10 or Aspect 11, wherein the solvent comprises toluene.

Aspect 13 is the method of any one of Aspects 10-12, wherein the purified trifluoroacetyl (TFAI) contains less than 2000 ppm iodine (I₂).

Aspect 14 is the method of any one of Aspects 10-13, wherein a first column includes a condenser and rectification section.

Aspect 15 is the method of Aspect 14, wherein the first column includes a reboiler and stripping section.

Aspect 16 is the method of any one of Aspects 10-15, wherein a second column includes a reboiler and stripping section.

Aspect 17 is the method of any one of Aspects 10-16, wherein the solvent may be recycled back to the feed stream.

Aspect 18 is a method for removing iodine (I₂) from trifluoroacetyl iodide (TFAI), the method comprising: adding a third component to a mixture of iodine (I₂) and trifluoroacetyl iodide (TFAI), wherein the third component is immiscible or nearly immiscible with iodine (I₂); heating the mixture to melt the iodine (I₂); allowing the mixture to settle into two layers; and separating the layers into a top and bottom layer; wherein the top layer comprises trifluoroacetyl iodide (TFAI) and the bottom layer comprises liquid iodine (I₂).

Aspect 19 is the method of Aspect 18, wherein the third component comprises trifluoroacetic acid.

Aspect 20 is the method of either Aspect 18 or Aspect 19, wherein the mixture is heated to 110° C. to 130° C.

Aspect 21 is the method of any one of Aspects 18-20, further comprising solidifying the liquid iodine (I₂).

Aspect 22 is the method of any one of Aspects 18-21, wherein the trifluoroacetyl (TFAI) contains less than 2000 ppm iodine (I₂).

What is claimed is:

1. A method for solvation and removal of an iodine (I₂)-containing species comprising the following steps:
providing a feedstock comprising trifluoroacetyl iodide (TFAI) and the iodine (I₂)-containing species;
adding a solvent to the feedstock stream to provide a mixture comprising the solvent, trifluoroacetyl iodide (TFAI) and the iodine (I₂)-containing species; and
passing the mixture to one or more columns to obtain a purified stream comprising trifluoroacetyl iodide (TFAI).

2. The method of claim 1, wherein the solvent comprises one or more of benzene, toluene, xylenes, mesitylene (1,3,5-trimethylbenzene), ethyl benzene, alkylated benzenes, dimethylformamide (DMF), dimethyl sulfoxide, (DMSO), imidazolium salts and caprolactamium hydrogen sulfate.

3. The method of claim 2, wherein the solvent comprises toluene.

4. The method of claim 1, further comprising:
passing the mixture to a first column to provide a first overhead product and a first bottoms product comprising the solvent, trifluoroacetyl iodide (TFAI), and at least an iodine (I₂)-containing species selected from HI₃ and iodine (I₂); and
passing the first bottoms product to a second column to provide a second overhead product comprising purified trifluoroacetyl iodide (TFAI) and a second bottoms product comprising the solvent and at least an iodine (I₂)-containing species selected from HI₃ and iodine (I₂); and passing the second bottoms product to a third column to provide a third overhead comprising the solvent and a third bottoms product comprising iodine (I₂) and the solvent.

5. The method of claim 4, wherein the purified trifluoroacetyl (TFAI) contains less than 2000 ppm iodine (I₂).

6. The method of claim 4, further comprising recycling the solvent.

7. The method of claim 1, further comprising:
passing the mixture first column to provide a first overhead product and a first bottoms product comprising the solvent, trifluoroacetyl iodide (TFAI), trifluoroacetic acid (TFA), and at least an iodine (I₂)-containing species selected from HI₃ and iodine (I₂);
passing the first bottoms product to a second column to provide a second overhead product comprising trifluoroacetyl iodide (TFAI) and a second bottoms product comprising the solvent, trifluoroacetic acid (TFA), high-boiling impurities, and at least an iodine (I₂)-containing species selected from HI₃ and iodine (I₂);
passing the second bottoms product a third column to provide a third overhead product comprising the solvent, trifluoroacetic acid (TFA), and high boiling impurities, and a third bottoms product comprising iodine (I₂);
passing the third overhead product to a fourth column to provide a fourth overhead product and a fourth bottoms product;
purging the fourth overhead product; and
recycling the fourth bottoms product back to the second column.

8. The method of claim 7, wherein the purified trifluoroacetyl (TFAI) contains less than 2000 ppm iodine (I₂).

9. The method of claim 7, further comprising recycling the solvent.

10. A method of removing iodine (I₂) from a stream comprising trifluoroacetyl iodide (TFAI), and at least an iodine (I₂)-containing species selected from iodine (I₂) and HI3, the method comprising:
providing a feed stream, a solvent, and at least an iodine (I₂)-containing species selected from iodine (I₂) and HI3; and
passing the feed stream to one or more columns to provide a purified trifluoroacetyl iodide (TFAI) product stream.

11. The method of claim 10, wherein the solvent comprises one or more of benzene, toluene, xylenes, mesitylene (1,3,5-trimethylbenzene), ethyl benzene, alkylated benzenes, dimethylformamide (DMF), dimethyl sulfoxide, (DMSO), imidazolium salts and caprolactamium hydrogen sulfate.

12. The method of claim 11, wherein the solvent comprises toluene.

13. The method of claim 10, wherein the purified trifluoroacetyl (TFAI) contains less than 2000 ppm iodine (I₂).

14. The method of claim 10, wherein a first column includes a condenser and rectification section.

15. A method for removing iodine (I₂) from trifluoroacetyl iodide (TFAI), the method comprising:
adding a third component to a mixture of iodine (I₂) and trifluoroacetyl iodide (TFAI), wherein the third component is immiscible or nearly immiscible with iodine;
heating the mixture to melt the iodine (I₂);
allowing the mixture to settle into two layers; and
separating the layers into a top and bottom layer; wherein the top layer comprises trifluoroacetyl iodide (TFAI) and the bottom layer comprises liquid iodine (I₂).
